(19) United States
(12) Patent Application Publication
(10) Pub. No.: US 2009/0095303 A1
(43) Pub. Date: Apr. 16, 2009

(54) Nasal prongs

(76) Inventors: Bruce Sher, Lighthouse Point, FL (US); Shara Hernandez, Davie, FL (US); Louis Javier Collazo, Ponpano Beach, FL (US)

Correspondence Address: MAIER & MAIER, PLLC, 1000 DUKE STREET, ALEXANDRIA, VA, 22314, US

(21) Appl. No.: 11/987,842

(22) Filed: Dec. 5, 2007

Related U.S. Application Data
(60) Provisional application No. 60/960,833, filed on Oct. 16, 2007.

(51) Int. Cl. A61M 15/08; A61B 19/00

(52) U.S. Cl. 128/207.18; 128/898

(57) ABSTRACT

A nasal prong for a ventilation interface can include a guide. The guide can be suitably disposed on portions of a nasal prong. One or more guides on a pair of nasal prongs may assist in operatively positioning and retaining the nasal prongs against or within a user's nares. In at least one exemplary embodiment, a guide can extend from the proximal end of a head portion of a nasal pillow. The guide may extend substantially upward from the nasal pillow. The guide may also be curved partially around a circumference of the nasal pillow. The guide can shroud a normally viewable portion of a user's nose when in use. The guide can abut an outer anatomy of a user's nose proximate a naris. As such, guides can cooperate with outer portions of the nose in which they contact to assist in positioning and retaining nasal pillows.

### CROSS REFERENCE TO RELATED APPLICATIONS

This application claims priority, under 35 U.S.C. §119(e), to U.S. Provisional Patent Application Ser. No. 60/960,833, filed Oct. 16, 2007, the disclosure of which is incorporated by reference herein in its entirety.

### BACKGROUND

Obstructive sleep apnea syndrome (commonly referred to as obstructive sleep apnea, sleep apnea syndrome, and/or sleep apnea) is a medical condition that includes repeated, prolonged episodes of cessation of breathing during sleep. During a period of wakefulness, the muscles of the upper part of the throat passage of an individual keep the passage open, thereby permitting an adequate amount of oxygen to flow into the lungs. During sleep, the throat passage tends to narrow due to the relaxation of the muscles. In those individuals having a relatively normal-sized throat passage, the narrowed throat passage remains open enough to permit an adequate amount of oxygen to flow into the lungs. However, in those individuals having a relatively smaller-sized throat passage, the narrowed throat passage prohibits an adequate amount of oxygen from flowing into the lungs. Additionally, a nasal obstruction, such as a relatively large tongue, and/or certain shapes of the palate and/or the jaw of the individual, further prohibit an adequate amount of oxygen from flowing into the lungs.

An individual having the above-discussed conditions can stop breathing for one or more prolonged periods of time (e.g., ten seconds or more). The prolonged periods of time during which breathing is stopped, or apneas, are generally followed by sudden reflexive attempts to breathe. The reflexive attempts to breathe are generally accompanied by a change from a relatively deeper stage of sleep to a relatively lighter stage of sleep. As a result, the individual suffering from obstructive sleep apnea syndrome generally experiences fragmented sleep that is not restful. The fragmented sleep results in one or more of excessive and/or inappropriate daytime drowsiness, headache, weight gain or loss, limited attention span, memory loss, poor judgment, personality changes, lethargy, inability to maintain concentration, and depression.

Other medical conditions can also prevent individuals, including adults and infants, from receiving an adequate amount of oxygen into the lungs. For example, an infant who is born prematurely can have lungs that are not developed to an extent necessary to receive an adequate amount of oxygen. Further, prior to, during and/or subsequent to certain medical procedures and/or medical treatments, an individual can be unable to receive an adequate amount of oxygen.

Under these circumstances, it is known to use a ventilation interface to apply a positive pressure to the throat of the individual, thereby permitting an adequate amount of oxygen to flow into the lungs. In known ventilation interfaces, oxygen and/or room air containing oxygen is delivered through the mouth and/or nose of the individual.

Existing types of positive pressure applied by the known ventilation interface include continuous positive airway pressure (CPAP), in which a positive pressure is maintained in the throat passage throughout a respiratory cycle, bi-level positive airway pressure (BiPAP), in which a relatively high positive pressure is maintained during inspiration and a relatively low positive pressure is maintained during expiration, and intermittent mechanical positive pressure ventilation (IPPV), in which a positive pressure is applied when apnea is sensed (i.e., the positive airway pressure is applied intermittently or non-continuously), automatic positive airway pressure (APAP), in which a positive pressure is automatically tuned to provide the minimum required to maintain an unobstructed throat passage on a breath-by-breath basis.

One conventional ventilation interface for the application of positive pressure includes a face mask that covers both the nose and the mouth. U.S. Pat. No. 4,263,908 (Mizerak) discloses a nasal cannula having oral gas delivery means incorporated therein adapted to increase efficiency in providing gas, such as oxygen to a patient. U.S. Pat. No. 6,123,071 (Berthon-Jones et al) discloses a combination mouth and nasal mask for assisted respiration or CPAP. At least one other ventilation interface is disclosed by U.S. Patent Application Publications Nos. 2006/0124131 (Chandran et al.) and 2006/0174887 (Chandran et al.). Other face masks include configurations that cover only the nose or only the mouth. Standard masks have air supplied under pressure and use headgear or harnesses to hold the mask on a user.

### SUMMARY

According to at least one exemplary embodiment, an article for a ventilation interface can include a nasal prong having a head portion and a body portion. A guide can extend from the nasal prong. The guide can be defined by a guide wall bounded by a guide edge. The guide can abut an outer anatomy of a wearer's nose when in use.

In another exemplary embodiment, an article for a ventilation interface can include a first nasal prong having a first guide and a second nasal prong having a second guide. A connector can connect the first nasal prong to the second nasal prong.

In yet another exemplary embodiment, a method of positioning and retaining a nasal prong can include positioning a nasal prong proximate a wearer's naris, cooperating a guide with outer portions of a wearer's nose and retaining the nasal prong proximate the wearer's naris.

### BRIEF DESCRIPTION OF THE FIGURES

Advantages of embodiments of the present invention will be apparent from the following detailed description of the exemplary embodiments thereof, which description should be considered in conjunction with the accompanying drawings in which:

FIG. 1A is a perspective view of an exemplary nasal pillow.

FIG. 1B is a rotated view of the exemplary nasal pillow.

FIG. 1C is another rotated view of an exemplary nasal pillow.

FIG. 1D is yet another rotated view of an exemplary nasal pillow.

FIG. 1E is still another rotated view of an exemplary nasal pillow.

FIG. 1F is a top view of an exemplary nasal pillow.

FIG. 1G is a bottom view of an exemplary nasal pillow.

FIG. 2A is a perspective view of an exemplary pair of nasal pillows.

FIG. 2B is a side view of an exemplary pair of nasal pillows.

FIG. 2C is an opposite side view of an exemplary pair of nasal pillows.

FIG. 2D is a front view of an exemplary pair of nasal pillows.

FIG. 2E is a rear view of an exemplary pair of nasal pillows.

FIG. 2F is a top view of an exemplary pair of nasal pillows.

FIG. 2G is a bottom view of an exemplary pair of nasal pillows.

FIG. 3A is a perspective view of another exemplary pair of nasal pillows.

FIG. 3B is a side view of another exemplary pair of nasal pillows.

FIG. 3C is an opposite side view of another exemplary pair of nasal pillows.

FIG. 3D is a front view of another exemplary pair of nasal pillows.

FIG. 3E is a rear view of another exemplary pair of nasal pillows.

FIG. 3F is a top view of another exemplary pair of nasal pillows.

FIG. 3G is a bottom view of another exemplary pair of nasal pillows.

FIG. 4A is a perspective view of another exemplary nasal pillow.

FIG. 4B is a top view of another exemplary nasal pillow.

FIG. 4C is a rotated view of another exemplary nasal pillow.

FIG. 4D is another rotated view of another exemplary nasal pillow.

FIG. 5A is a perspective view of yet another exemplary nasal pillow.

FIG. 5B is a top view of yet another exemplary nasal pillow.

FIG. 5C is a rotated view of yet another exemplary nasal pillow.

FIG. 5D is another rotated view of yet another exemplary nasal pillow.

FIG. 6 is an exploded view of an exemplary respiration assist mask.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the terms “embodiments of the invention,” “embodiments” or “invention” do not require that all embodiments of the invention include the discussed feature, advantage or mode of operation.

Embodiments can be designed to cooperate with nearly any ventilation interface/respiration assist mask that has nasal prongs for engagement with a user's nares. For examples, embodiment can be designed to cooperate with nasal cannulas and “hybrid” masks (i.e. those masks having an oral cavity and nasal prongs) of various styles and shapes, as will be readily recognized by those having ordinary skill in the art.

Accordingly, embodiments described below and the principles thereof may be applied to, for example, ventilation interfaces disclosed in U.S. Patent Application Publication Nos. 2006/0124131 (Chandran et al.), 2006/0174887 (Chandran et al.), and 2007/0272249 (Chandran et al.), the disclosures of which are incorporated by reference herein in their entireties.

Embodiments described below and the principles thereof may also be applied to, for example, ventilation interfaces disclosed in U.S. Pat. Nos. 6,595,215, 6,776,162, 6,807,967, 6,994,089, 6,997,187, 7,000,613, 7,047,974, 7,059,328, 7,188,624, and 7,191,781, the disclosures of which are incorporated by reference herein in their entireties.

Nasal prongs can generally be separated into two types; nasal pillows and nasal inserts. Nasal pillows typically abut against the openings of a user's nares when in use and may not be inserted substantially within the nasal passages. Conversely, nasal inserts are typically positioned within the nasal passages of a user and may or may not abut against the nasal openings. Embodiments and the principles thereof are contemplated for any nasal prong and the like, as will be readily recognized by one having ordinary skill in the art. Nevertheless, for illustrative purposes in a non-limiting manner, exemplary embodiments are described below in reference to nasal pillows.

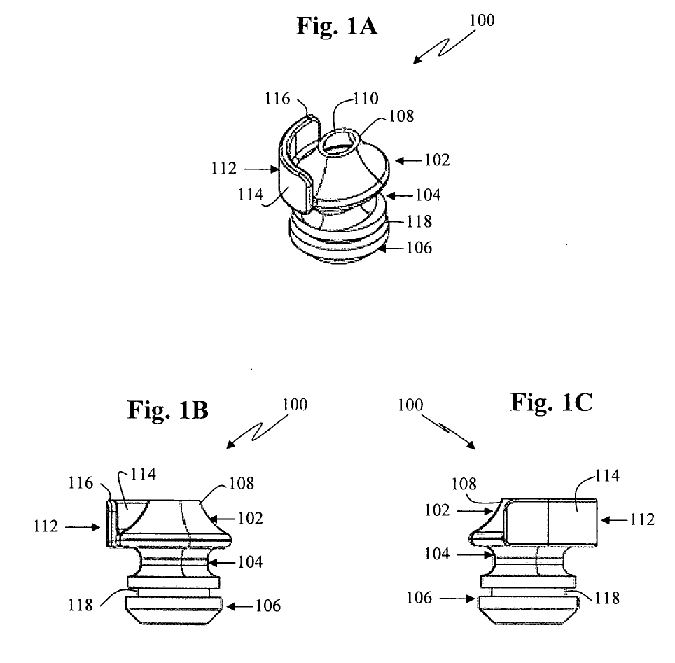

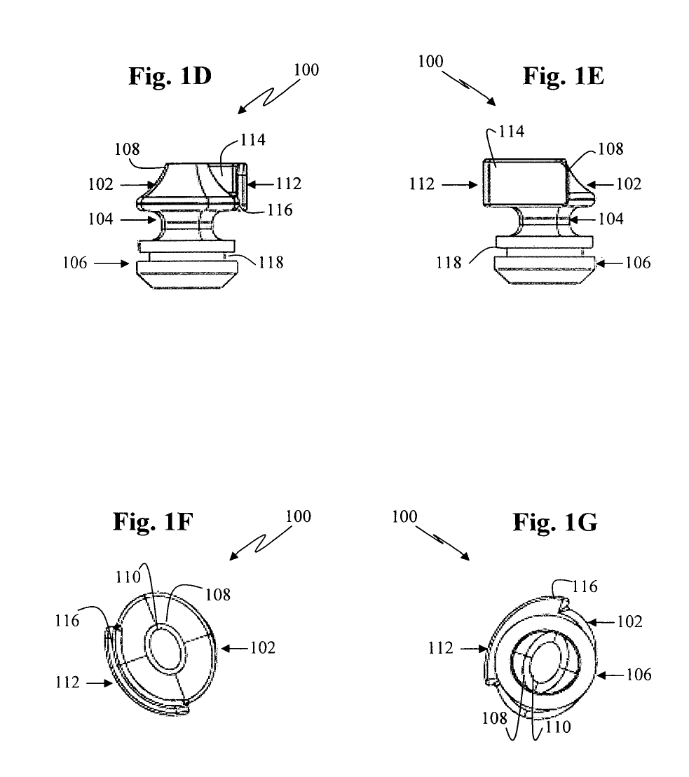

Referring to FIGS. 1A-1G, nasal pillow 100 can have head portion 102, body portion 104 and flange portion 106. Head portion 102 can extend from a first end of body portion 104. Flange portion 106 can extend from a second end of body portion 104.

Nasal pillow 100 and portions thereof can be flexible, rigid or any combination thereof. Nasal pillow 100 and portions thereof can be formed integrally or non-integrally. In at least one exemplary embodiment, nasal pillow 100 can be formed from an elastomeric material. For example, nasal pillow 100 can be integrally formed from a silicone elastomer using conventional molding processes known to one having ordinary skill in the art.

Head portion 102 can be formed in various shapes, such as the volcano style shown or any other style known to one having ordinary skill in the art. The distal end of head portion 102 can form distal tip 108. Distal tip 108 can have opening 110, which may form a gas exit from a hollow channel defined within nasal pillow 100. Distal tip 108 can be abutted against and may breach a nasal opening of a user when positioned for use. Distal tip 108 can deformably contact portions of a naris of a user. Distal tip 108 can thus sealingly engage a naris of a user. Distal tip 108 may form a gas-tight seal with a naris of a user for receiving a breathable gas, for example, under positive pressure.

Guide 112 can be suitably disposed on any part of nasal pillow 100. Guide 112 may assist in operatively positioning and retaining head portion 102, particularly, proximate distal tip 108, with a naris of a user. Guide 112 may reduce accidental dislodgements of nasal pillow 100 with a naris of a user.

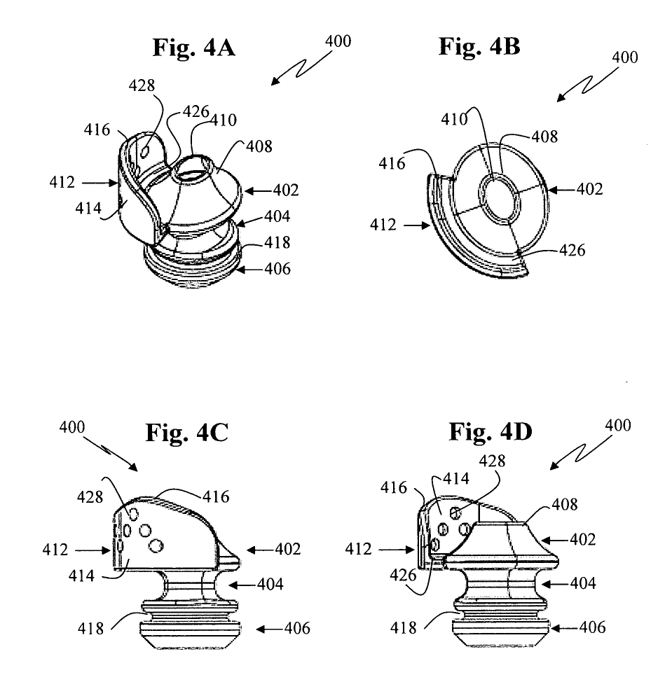

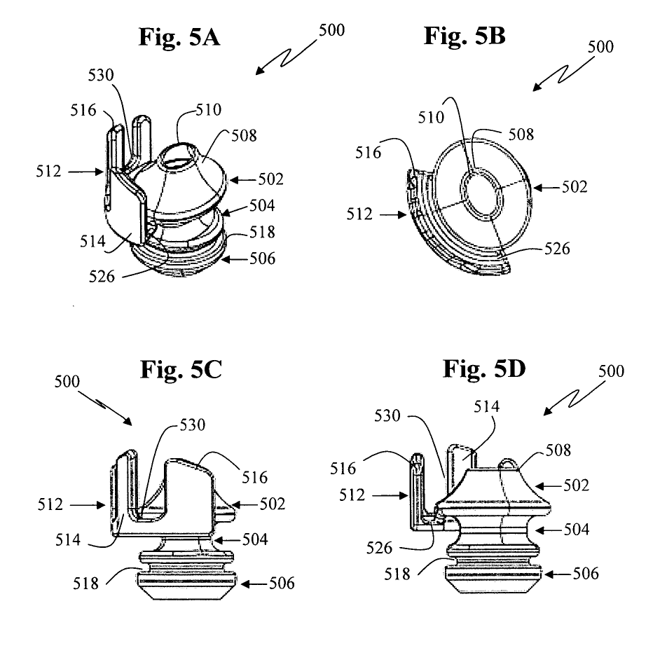

Guide 112 can extend from the proximal end (e.g., the base) of head portion 102 or proximate thereto. As shown, in at least one exemplary embodiment, guide 112 can extend substantially upward and may extend somewhat outwardly from a position proximate the proximal end of head portion 102. Guide 112 may extend substantially straight in an upward direction. Alternatively, guide 112 may extend upward at any angle and/or may extend with curvature. Guide 112 may extend upwards so as to be at substantially the same height as distal tip 108. Alternatively, guide 112 may extend upwards so as to be above or below distal tip 108 (see, e.g., FIGS. 4A-4D and 5A-5D).

Guide 112 can be defined by guide wall 114 and guide edge 116. Guide wall 114 can be bounded, at least in part, by guide edge 116. Guide wall 114 may be curved around a circumference of nasal pillow 100 in whole or in part. Alternatively, guide wall 114 may not be curved. Guide wall 114 may be continuous (as shown) or, alternatively, non-continuous (e.g., segmented). One or more apertures (not shown) may be defined in guide wall 114 (see, e.g., FIGS. 4A-4D). Apertures can be in the form of slots, perforations and like openings. Apertures may be called for in order to reduce the weight of guide wall 114 and/or in order to reduce moisture on outer portions of a user's nose that may contact guide wall 114.

As shown, in at least one exemplary embodiment, guide wall 114 can be continuously curved less than halfway around the circumference of the base of head portion 102. Guide 112 can shroud a normally viewable portion of a user's nose when in use. Guide 112 can abut an outer anatomy of a user's nose proximate a naris of a user. Particularly, the inner side of guide wall 114 can border portions of the outer anatomy of a user's nose laterally proximate a naris of a user. As such, guide 112 can cooperate with outer portions of a user's nose in which it contacts (continuously or intermittently) to assist in positioning and retaining nasal pillow 100 when in use.

Guide edge 116 and portions thereof may be rounded. Rounded guide edge 116 (or portions thereof) may increase user comfort. Guide 112, as a whole, may be contoured in shape, which may also increase user comfort. For example, the top portion and side portions of guide 112 can vary to produce a configuration that can have one or more curved surfaces (see, e.g., FIGS. 4A-4D). Alternatively, guide 112 can be substantially linear and guide edge 116 can be substantially flat. Also, as previously stated, guide wall 114 can be curved in any direction or be substantially linear.

Similarly, to nasal pillow 100 as a whole, guide 112 and portions thereof can be flexible, rigid or any combination thereof. Guide 112 can be formed integrally or non-integrally with nasal pillow 100 (or any portions thereof). Guide 112 can be retrofittablely disposed on preexisting nasal prongs. In at least one exemplary embodiment, guide 112 can be formed from an elastomeric material. For example, guide 112 can be integrally formed with nasal pillow 100 from a silicone elastomer using conventional molding processes known to one having ordinary skill in the art.

Still referring to FIGS. 1A-1G, body portion 104 can be a variety of shapes. As shown, body portion 104 can be indented. Indented body portion 104 may act to compress when nasal pillow 100 is positioned against a naris of a user. This compression may allow for better fitment and comfort of nasal pillow 100, as well as provide for an adjustment of height. Additionally, indented body portion 104 can allow for lateral movements of nasal pillow 100. Thus, comfort may be increased and additional adjustments may be made to the fitment of nasal pillow 100.

Nasal pillow 100 can have a variety of coupling structures extending from the second end of body 104, including various types of flanges and the like known to one having ordinary skill in the art. In at least one exemplary embodiment, flange portion 106 can extend from body portion 104 for coupling nasal pillow 100 with a receiving hole defined on a portion of a ventilation interface.

Flange portion 106 can have one or more grooves 118 (one groove 118 as shown) for coupling with a receiving hole of a ventilation interface. The distal end of flange portion 106 can be deformable. The distal end portion of flange portion 106 can be forced through a receiving hole of a ventilation interface. As a result, groove 118 can be sealingly mated within the receiving aperture. Flange portion 106 may thus form a gas-tight seal at the receiving aperture and can allow for the provision of breathable gas, for example, under positive pressure, through nasal pillow 100 without leakage or contamination from the ambient environment at the seal when in use.

In other embodiments, flange portion 106 may not be necessary. For example, nasal pillow 100 can be integrally molded with another portion of a ventilation interface, such as with a cushion or a shell of a “hybrid” mask. In at least one additional embodiment, nasal pillow 100 can be integrally molded with a nasal cannula and like ventilation interfaces having nasal prongs known to one having ordinary skill in the art.

In further embodiments, an additional component can be used to increase any clamping action of guide 112. For example, a wire, a strip (e.g., metal or plastic), an elastic strap or like stiffening/resiliency-adding agents can be molded into, attached to the outside of or otherwise attached to guide 112. Such agents can be used to provide more force against portions of a user's nose trapped between guide 112 and head portion 104. Such agents may further decrease incidents of dislodgment of the nasal pillow 100 from a naris of a user when in use.

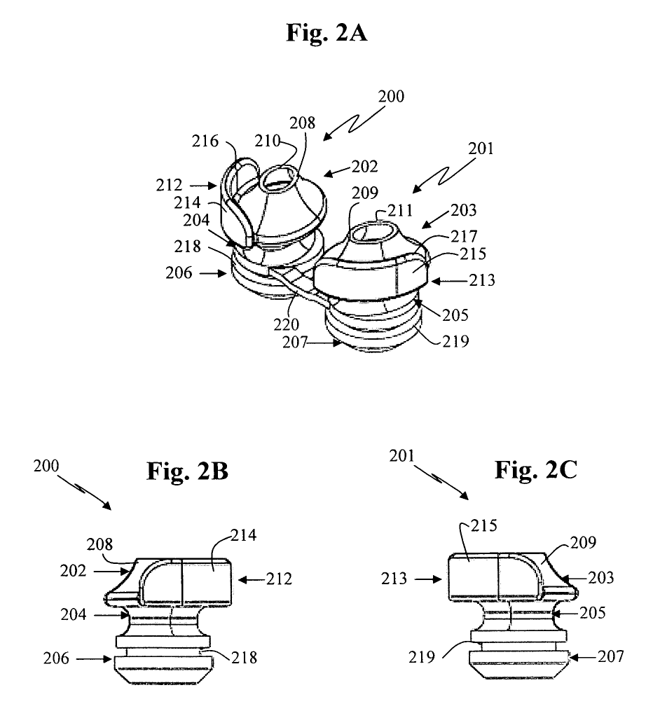

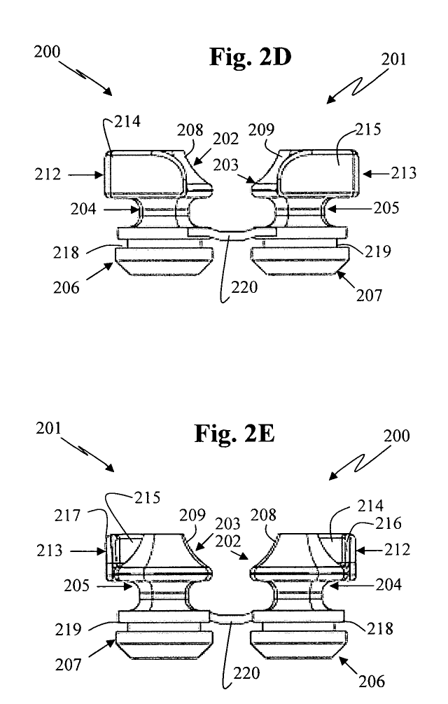

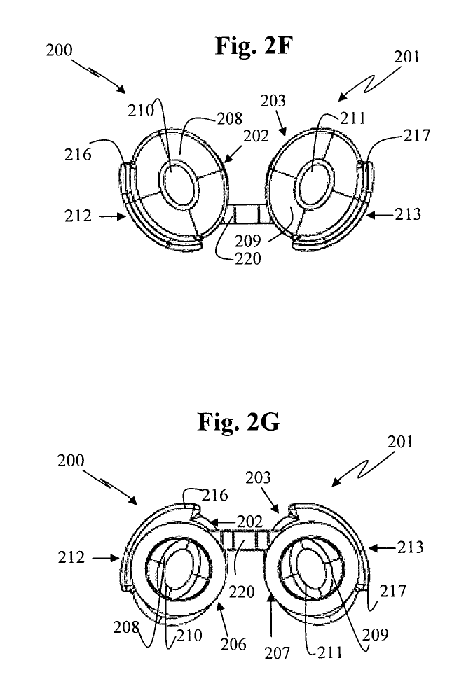

Referring to FIGS. 2A-2G, a pair of nasal pillows 200, 201, substantially similar to nasal pillow 100 of FIG. 1A-1G, can be coupled by connector 220 in accordance with at least one exemplary embodiment. Nasal pillow 200 can include head portion 202, body portion 204, flange portion 206, distal tip 208, opening 210, guide 212, guide wall 214, guide edge 216 and groove 218. Likewise, nasal pillow 201 can include head portion 203, body portion 205, flange portion 207, distal tip 209, opening 211, guide 213, guide wall 215, guide edge 217 and groove 219. Any further redundant description of like elements does not bear repeating here.

Connector 220 can be formed out of any material and may, optionally, be formed out of the same material as nasal pillows 200, 201. Connector 220 can be permanently attached or removablely attached with nasal pillows 200, 201. Connector 220 may function to prevent the rotation of nasal pillows 200, 201 when they are respectively engaged in receiving holes of a ventilation interface. Additionally, connector 220 may act to retain nasal inserts 200, 201 in a desired position.

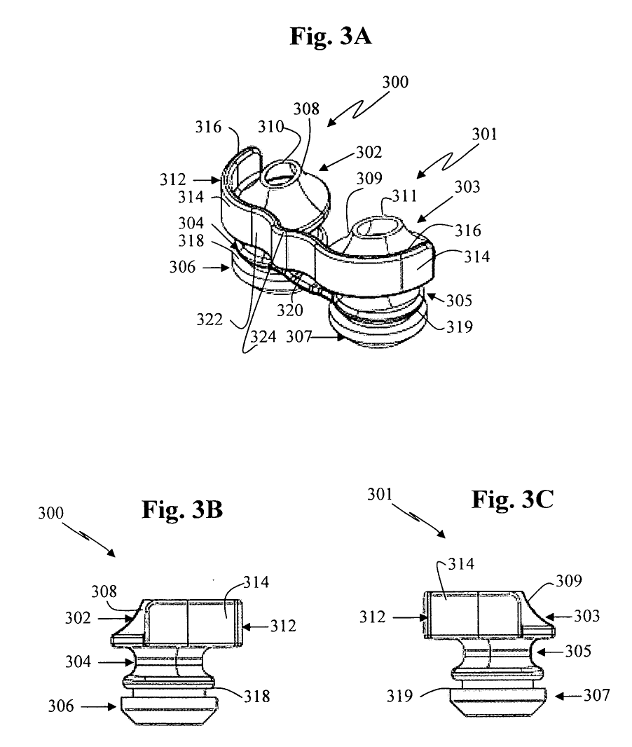

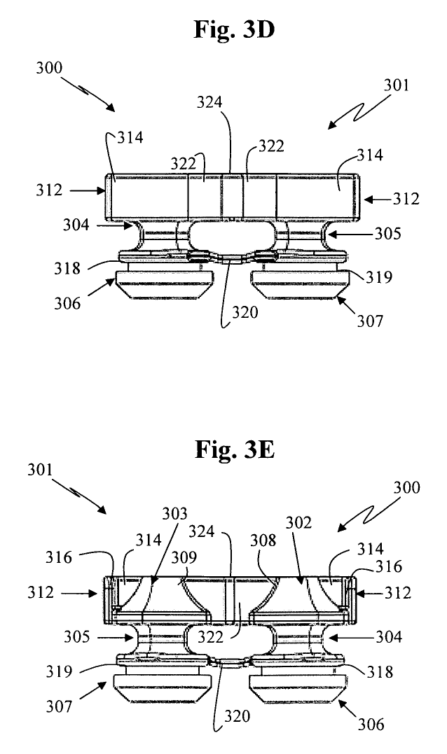

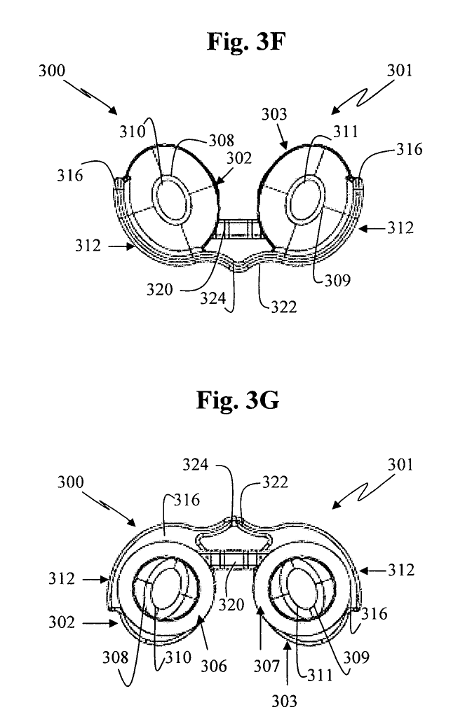

Referring to FIGS. 3A-3G, a guide in accordance with at least one exemplary embodiment is disclosed. Nasal pillows 300, 301 can be substantially similar, to nasal pillows 200, 201 of FIGS. 2A-2G with the exception that exemplary independent guides 212, 213 can be substituted by exemplary guide 312. Otherwise, nasal pillow 300 can include head portion 302, body portion 304, flange portion 306, distal tip 308, opening 310 and groove 318. Likewise, nasal pillow 301 can include head portion 303, body portion 305, flange portion 307, distal tip 309, opening 311 and groove 319. Optionally, connector 320 can connect nasal pillow 300 to nasal pillow 301. Alternatively, connector 320 may be excluded and guide 312 can function as a connector. Any further redundant description of such like elements does not bear repeating here.

Guide 312 can be suitably disposed on parts of nasal pillows 300, 301. Guide 312 may assist in operatively positioning and retaining head portions 302, 303 with a user's nares. Guide 312 may reduce accidental dislodgements of both nasal pillows 300, 301.

Portions of guide 312 can extend from the proximal ends (or proximate thereto) of head portions 302, 303. As shown, in at least one exemplary embodiment, guide 312 can extend substantially upward and may extend somewhat outwardly from positions proximate the proximal ends of head portions 302, 303. Guide 312 may extend substantially straight in an upward direction. Alternatively, guide 312 may extend upward at any angle and/or may extend with curvature.

Guide 312 may extend upwards so as to be at substantially the same height as distal tips 308, 309. Alternatively, guide 312 may extend upwards so as to be above or below distal tips 308, 309.

Guide 312 can be defined by guide wall 314 and guide edge 316. Guide wall 314 can be bounded, at least in part, by guide edge 316. Portions of guide wall 314 and guide edge 316 can form guide bridge 322. Guide bridge 322 can bridge the gap between nasal pillow 300 and nasal pillow 301. Guide bridge 322 may include notch 324 for receiving a portion of a user's nose, such as the tip of a user's nose. Alternatively, guide bridge 322 can be contoured in various ways as will be readily recognized by one having ordinary skill in the art.

Guide 312, as a whole, may be contoured in shape, which may increase user comfort. For example, the top portion and side portions of guide 312 can vary to produce a configuration that can have one or more curved surfaces. Alternatively, guide 312 can be substantially linear. Also, guide wall 314 can be curved in any direction or can be substantially linear.

Guide wall 314 may be curved around a circumference (in whole or in part) of nasal pillow 300 and may be curved around a circumference (in whole or in part) of nasal pillow 301. Alternatively, guide wall 314 may not be curved. As shown, guide 312 can be curved less than halfway around a circumference of head portion 302 and less than halfway around a circumference of head portion 303 (e.g., at the respective bases thereof).

Guide 312 may be continuous (as shown) or, alternatively, non-continuous (e.g., segmented). One or more apertures (not shown) may also be defined in guide 312. Apertures can be in the form of slots, perforations and like openings.

Guide 312 can shroud a normally viewable portion of a user's nose when in use. Guide 312 can abut an outer anatomy of a user's nose proximate the user's nares. As such, guide 312 can cooperate with outer portions of a user's nose in which it contacts (continuously or intermittently) to assist in positioning and retaining nasal pillows 300, 301 when in use.

Guide edge 316 and portions thereof may be rounded. Rounded guide edge 316 (or portions thereof) may increase user comfort. Alternatively, guide edge 316 can be substantially flat.

Similar to nasal pillows 300, 301 as a whole, guide 312 and portions thereof can be flexible, rigid or any combination thereof. Guide 312 can be formed integrally or non-integrally with nasal pillows 300, 301 (or any portion thereof). Guide 312 can be retrofittablely disposed on preexisting nasal prongs. In at least one exemplary embodiment, guide 312 can be formed from an elastomeric material. For example, guide 312 can be integrally formed with nasal pillows 300, 301 from a silicone elastomer using conventional molding processes known to one having ordinary skill in the art.

Referring to FIGS. 4A-4D, nasal pillow 400 is shown in accordance with at least one other exemplary embodiment. Without reference to guide 412, nasal pillow 400 can be substantially similar to nasal pillow 100 of FIGS. 1A-1G. Similar to nasal pillow 100, nasal pillow 400 can include head portion 402, body portion 404, flange portion 406, distal tip 408, opening 410 and groove 418. Any further redundant description of such like elements does not bear repeating here.

As shown, guide 412 can extend from the proximal end (e.g., the base) of head, portion 402 or proximate thereto. Spacer portion 426 of guide 412 can extend guide 412 in a substantially horizontal direction away from head portion 402. Spacer portion 426 can be curved, straight and any combination thereof. Spacer portion 426 may have one or more grooves, indentations, notches, pits and like elevated and depressed regions (see, e.g., FIGS. 5A-5D).

At the outer end of spacer portion 426, guide 412 can extend substantially upward. Guide 412 may extend substantially straight in an upward direction. Alternatively, guide 412 may extend upward at any angle and/or may extend with curvature. In at least one exemplary embodiment, guide 412 can extend upwards beyond distal tip 408.

Similar to guide 112, guide 412 can be defined by guide wall 414 and guide edge 416. Guide wall 414 can be bounded, at least in part, by guide edge 416. Guide wall 414 may be curved around a circumference of nasal pillow 400 in whole or in part. Also similar to guide 112, guide wall 414 can be continuously curved less than halfway around the circumference of the base of head portion 402. Likewise, guide edge 416 and portions thereof may be rounded and/or substantially flat.

One or more apertures 428 can be defined in guide wall 414. Apertures 428 can be in the form of slots, perforations and like openings. Apertures 428 may act as weight reducers and/or as elements directed towards providing breathability to guide 412.

Guide 412, as a whole, can be contoured in shape. The contoured shape of guide 412 may increase user comfort. For example, the top portion and side portions of guide 412 can vary to produce a configuration that can have one or more curved surfaces. As shown, guide 412 can have a top portion and side portions formed to follow a generally sweeping curve (i.e. a single curvature pattern).

Similar to guide 112, guide 412 and portions thereof can be flexible, rigid or any combination thereof. Guide 412 can be formed integrally or non-integrally with nasal pillow 400 (or any portions thereof). Guide 412 can be retrofittablely disposed on preexisting nasal prongs. In at least one exemplary embodiment, guide 412 can be formed from an elastomeric material. For example, guide 412 can be integrally formed with nasal pillow 400 from a silicone elastomer using conventional molding processes known to one having ordinary skill in the art.

Referring to FIGS. 5A-5D, nasal pillow 500 is shown in accordance with at least one exemplary embodiment. Without reference to guide 512, nasal pillow 500 can be substantially similar to nasal pillow 100 of FIGS. 1A-1G. Similar to nasal pillow 100, nasal pillow 500 can include head portion 502, body portion 504, flange portion 506, distal tip 508, opening 510 and groove 518. Any further redundant description of such like elements does not bear repeating here.

Guide 512 can extend from the top end of body portion 504 proximate the base of head portion 502. Guide 512 may also be conjunctively connected to portions of the base of head portion 502. Spacer portion 526 of guide 512 can extend guide 512 in a substantially horizontal direction away from body portion 504 (and head portion 502).

Spacer portion 526 can be curved, straight and any combination thereof. Spacer portion 526 may have one or more grooves, indentations, notches, pits and like elevated and depressed regions. As shown in FIG. 5D, spacer portion 526 can have a groove, which may be centrally defined within the width of spacer portion 526 and may traverse the length of spacer portion 526.

At the outer end of spacer portion 526, guide 512 can extend substantially upward. Guide 512 may extend substantially straight in an upward direction. Alternatively, guide 512 may extend upward at any angle and/or may extend with curvature. In at least one exemplary embodiment, guide 512 can extend upwards beyond distal tip 508.

Similar to guide 112, guide 512 can be defined by guide wall 514 and guide edge 516. Guide wall 514 can be bounded, at least in part, by guide edge 516. Guide wall 514 may be curved around a circumference of nasal pillow 500 in whole or in part. Also similar to guide 112, guide wall 514 can be continuously curved less than halfway around the circumference of the base of head portion 502. Likewise, guide edge 516 and portions thereof may be rounded and/or substantially flat.

One or more spaces 530 can be defined in guide wall 514. Spaces 530 can be of various shapes and sizes. Spaces 530 can provide pseudo-segmentation (i.e. appearance that guide 512 is non-continuous). Like apertures 428, spaces 530 may act as weight reducers and/or as elements directed towards providing breathability to guide 512. Also, spaces 530 may provide contoured features for increasing user comfort. Moreover, guide 512, as a whole, can be contoured in shape. The contoured shape of guide 512 may also increase user comfort.

Similar to guide 112, guide 512 and portions thereof can be flexible, rigid or any combination thereof. Guide 512 can be formed integrally or non-integrally with nasal pillow 500 (or any portions thereof). Guide 512 can be retrofittablely disposed on preexisting nasal prongs. In at least one exemplary embodiment, guide 512 can be formed from an elastomeric material. For example, guide 512 can be integrally formed with nasal pillow 500 from a silicone elastomer using conventional molding processes known to one having ordinary skill in the art.

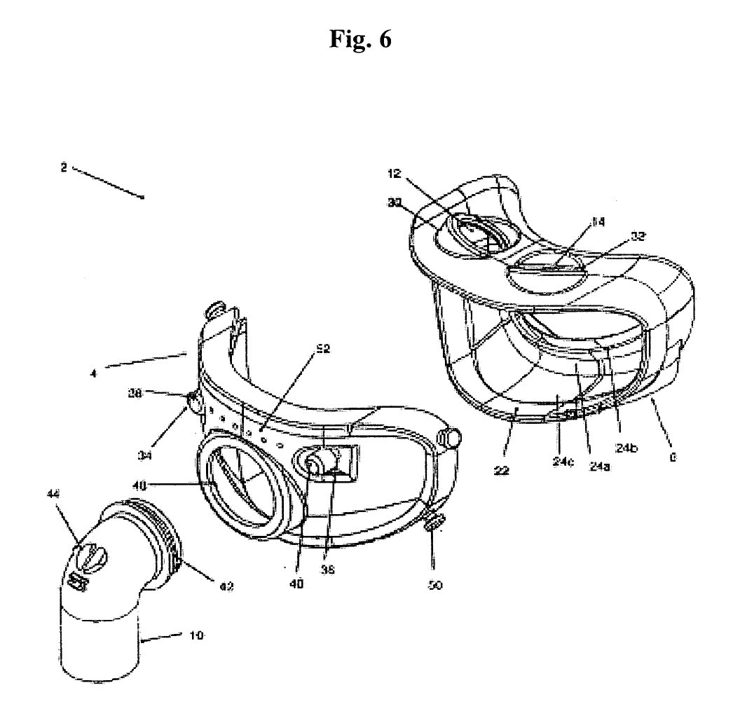

FIG. 6 shows an exploded view of an exemplary embodiment of a ventilation interface/respiration assist mask that can be used with any of the exemplary nasal pillows described above. Respiration assist mask 2 can have several separable components, such as mask shell 4, cushioned facial interface 6 and gas supply tube 10. Supply tube 10 can be connected to mask shell 4 in order for an input gas to be supplied to the device. Facial interface 6 can be joined with mask shell 4. Facial interface 6 can also accept a pair of nasal pillows through receiving holes 12 and 14. The respiration assist mask 2 can then be positioned over the mouth of a user such that facial interface 6 forms an airtight seal over the mouth of the user. Additionally, facial interface 6 can form a seal against the upper and lower lips of the user. When respiration assist mask 2 is positioned over the mouth of a user, the user can position the nasal pillows proximate the nares of a user. When the nasal pillows are positioned proximate the nares of a user, an airtight seal can be formed.

Facial interface 6 can provide an airtight seal against the face of a user. Additionally, facial interface 6 can act as a cushion against the face of a user. Similar to the removable cushion disclosed in, for example, U.S. Pat. No. 6,595,214, facial interface 6 can act as a removable cushion that attaches to a mask shell. Facial interface 6 can also act to form a seal against an upper and/or lower lip of a user.

Additionally, facial interface 6 can include chin flap 22. When respiration assist mask 2 is placed on the face of a user, chin flap 22 can be positioned under the chin of the user. Chin flap 22 may provide additional sealing against the face of a user. Additionally, chin flap 22 may act to provide additional comfort for a user. Chin flap 22 may also act to limit the movement of the lower jaw of a user.

Facial interface 6 can have multiple membranes 24a, 24b and 24c (collectively “membranes 24”). Membranes 24 can serve to provide additional seals against the face of a user. For example, membranes 24, and specifically membrane 24a, can seal against an upper and/or lower lip of a user who is wearing respiration assist mask 2. Membrane 24a can be formed to be thinner than membrane 24b. Thus, membrane 24a can adhere to facial contours and fill small facial gaps as it can be a thin, flexible material. Additionally, membrane 24b can be thicker than membrane 24a to provide auxiliary sealing against the face of a user and provide structural support for the device. For example, membrane 24a can be made of any suitable material, for example silicone, and may be approximately 0.020″ thick. Membrane 24b can also be made of any suitable material, for example silicone, and may have a thickness of approximately 0.050″. Still other parts of facial interface 6, for example 24c, may have a thickness of approximately 0.100″. This thickness can extend around the periphery of that portion of the device.

Also, membranes 24 can work in conjunction with chin flap 22 to provide additional sealing capabilities. As stated previously, chin flap 22 may act to limit the movement of the lower jaw of a user. Nevertheless, chin flap 22 can have some elasticity, which allows a user wearing respiration assist mask 2 to move their jaw and, for example, open their mouth. In the event of this happening, membrane 24a, which also can be elastic, can stretch upper portion of the lower jaw of the user, thus maintaining the seal between the interface and a wearer's face. Membrane 24b, which can also be elastic, can then stretch against the bottom portion of the mouth of the user, thus maintaining an airtight seal between facial interface 6 and the face of a user.

Moreover, movement of the lower jaw of a user will not break the airtight seal of respiration assist mask 2 against the face of a user or dislodge the nasal pillows, which can be positioned against the nares of a user. When the mouth of a user wearing the mask opens, chin flap 22 can allow facial interface 6 to stretch. For example, if a user were to open their mouth, the lower jaw of the user could move against chin flap 22, but may remain in contact with chin flap 22 as it stretches. Thus, when facial interface 6 stretches, membranes 24 can remain sealed against the moving face of the user.

Facial interface 6 may have contoured surfaces around receiving holes 12 and 14. These contoured surfaces can work in conjunction with the flange portions of the nasal pillows. Contoured surface 30 and contoured surface 32 can act to hold a pair of nasal pillows, respectively, in a position that allows for an airtight seal to be formed between the nasal pillows and the nares of a user wearing respiration assist mask 2. Also, contoured surfaces 30 and 32 can act to provide an airtight seal between a pair of nasal pillows, respectively and facial interface 6. In a further embodiment, contoured surfaces 30 and 32 can act to angle a pair of nasal pillows; respectively, towards each other and thus orientate them to be better positioned proximate the nares of a user.

In another exemplary embodiment shown in FIG. 6, auxiliary ports 34 and 36 can be positioned on mask shell 4. Auxiliary ports 34 and 36 can be positioned on an upper portion of mask shell 4 and can project outwardly. Additionally, when they are not being otherwise utilized, auxiliary ports 34 and 36 can be capped with coverings 38 and 40, respectively. Auxiliary ports can be used, for example, to connect to outside devices for the purposes of measuring oxygen or carbon dioxide levels, pressure, or to connect to any other outside device to provide measurements, readings or additional inputs. Alternatively, auxiliary ports 34 and 36 can be utilized as exhaust ports to release gas from the interior portion of mask shell 4. Removable coverings 38 and 40 can act to prevent the release of gas from respiration assist mask 2 and maintain the airtight seal within the device.

Mask shell 4 can also have a design such that it can accept and seal with facial interfaces of various sizes. In one exemplary embodiment, facial interface 6 can be made to have different size or shape cushions or have a different sealing area. Different size facial interfaces can maintain a similar size or shape membrane to connect with mask shell 4, however. Different size facial interfaces may be made out of a material that stretches, so as to allow for an airtight seal to be formed between varying sizes of facial interface and mask shell 4.

FIG. 6 also shows input gas tube 10, which can be formed in an elbow shape or any other shape which can attach to mask shell 4. Input gas tube can be used to deliver any type of gas or aerosol and can be used in any type of respiration application, such as CPAP or BiPAP applications. Input gas tube 10 can have connection portion 42 which can be used to connect input gas tube 10 to mask shell 4 through the use of receiving hole 48. Connection portion 42 can be threading, allowing input gas tube 10 to be screwed into receiving hole 48 or any other connection and sealing mechanism, such as a clip or a clasp. Input gas tube 10 can also have valve 44 disposed on its surface. Valve 44 can be coupled with a flap, which is closed in an airtight seal when ventilation gas is being passed through input gas tube 10. However, if there is no gas being inputted through tube 10, the flap will open, allowing outside air to enter respiration assist mask 2.

Respiration assist mask 2 can be worn on the face of a user with any of a variety of types of headgear (not shown). The headgear can attach to respiration assist mask 2 through the use of headgear attachment posts 50. Attachment posts 50 can be positioned at various portions of mask shell 4, for example at the top and bottom of either side face 4. The headgear may have female connectors that allow for the headgear to be securely fastened to male attachment posts 50. Alternatively, the headgear may have looped ends that securely fit around attachment posts 50. Additionally, any other known type of attachments or posts can be used to securely attach headgear to respiration assist mask 2 in such as manner as to provide for the comfort of a user and allow for an airtight seal to be formed between the face of a user and respiration assist mask 2.

Any of the above embodiments may be utilized in any of a variety of respiration or respiration assist devices and are not limited to respiration assist masks. The various nasal pillows may be utilized in any of a variety of devices, including but not limited to respiration assist masks, nasal cannulas, ventilation masks, underwater breathing apparatuses, and other type of device capable of delivering breathable gas or aerosol.

The foregoing description and accompanying drawings illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. An article for a ventilation interface, comprising:
a nasal prong having a head portion and a body portion; and
a guide extending from the nasal prong, the guide is defined by a guide wall and a guide edge, the guide wall bounded by the guide edge, wherein the guide is configured to abut an outer anatomy of a wearer's nose when in use.

2. The article of claim 1, wherein the nasal prong is a nasal pillow.

3. The article of claim 1, wherein the guide extends from the nasal prong proximate the head portion.

4. The article of claim 1, wherein the guide is curved less than all of the way around a circumference of the head portion.

5. The article of claim 1, wherein the guide wall is continuous.

6. The article of claim 1, wherein the guide wall has one or more apertures.

7. The article of claim 1, wherein at least a portion of the guide edge is rounded.

8. The article of claim 1, wherein the guide is contoured.

9. The article of claim 1, wherein the guide is segmented.

10. The article of claim 1, wherein the guide has pseudo-segmentation.

11. The article of claim 1, wherein the guide is formed integrally with the nasal prong.

12. The article of claim 1, wherein the guide is formed from a silicone elastomer.

13. The article of claim 1, wherein the guide extends substantially upward from a position proximate a base of the head portion.

14. The article of claim 13, wherein the guide extends outwardly from the position proximate the base of the head portion.

15. The article of claim 13, wherein the guide extends substantially upward to substantially the same height as a distal tip of the head portion.

16. The article of claim 1, further comprising:
a spacer portion extending the guide away from the nasal prong.

17. The article of claim 1, further comprising:
a flange portion extending from the body portion of the nasal prong.

18. An article for a ventilation interface, comprising:
a first nasal prong having a first guide;
a second nasal prong having a second guide: and
a connector connecting the first nasal prong to the second nasal prong.

19. The article of claim 18, further comprising:
a guide bridge connecting the first guide to the second guide, wherein the guide bridge also serves as a second connector for connecting the first nasal prong to the second nasal prong.

20. The article of claim 18, wherein the connector is a guide bridge, the guide bridge connecting the first guide to the second guide.

21. A method of positioning and retaining a nasal prong, comprising:
positioning a nasal prong proximate a naris;
cooperating a guide with one or more outer portions of a nose; and
retaining the nasal prong proximate the naris.
